(19) United States
(12) Patent Application Publication
(10) Pub. No.: US 2021/0386505 A1
(43) Pub. Date: Dec. 16, 2021

(54) BACTERIAL BIOFILM STAINING DEVICE AND METHODS OF USE

(71) Applicant: University of Utah Research Foundation, Salt Lake City, UT (US)

(72) Inventors: Jeremy D. Shaw, Salt Lake City, UT (US); Nicholas N. Ashton, Salt Lake City, UT (US); Dustin L. Williams, Salt Lake City, UT (US)

(21) Appl. No.: 17/328,578

(22) Filed: May 24, 2021

Related U.S. Application Data
(60) Provisional application No. 63/028,735, filed on May 22, 2020.

(51) Int. Cl. A61B 90/00; C12Q 1/04

(52) U.S. Cl. CPC A61B 90/39; C12Q 1/04; A61B 2090/3987; A61B 2090/395; C12Q 2304/18

(57) ABSTRACT

Methods and devices for removing non-viable tissue at a treatment situs are described. The method can include administering a selective biofilm stain dye to the treatment situs at a dilution that selectively stains non-viable tissue compared to viable tissue at a selectivity ratio that provides a visually identifiable separation between the non-viable tissue and the viable tissue. The selective biofilm stain dye is diluted in water to a concentration of from about 0.001 mg/mL to about 0.5 mg/mL. The method can also include visually identifying the non-viable tissue, and removing the non-viable tissue based on the visual identification.

A biofilm staining device can include an applicator having a dispense tip, a rupture lever adapted to rupture a rupturable capsule and release a selective biofilm stain dye, and a housing sized to retain the rupturable capsule and adapted to allow released selective biofilm stain dye to flow to the dispense tip.

### RELATED APPLICATION

This application claims the benefit of U.S. Provisional Patent Application Ser. No. 63/028,735, filed on May 22, 2020, the entire contents of which are incorporated herein by reference.

### GOVERNMENT INTEREST

None.

### FIELD OF THE INVENTION

The present disclosure relates to biofilm compounds and bacterio-physiology research. Therefore, the present disclosure relates generally to the fields of biology, cell physiology, immunology, and material science.

### BACKGROUND

Bacterial biofilms pose a challenge in treating hardware-associated infections. Biofilms—which are mostly invisible to the naked eye—provide bacteria protection against antimicrobial agents and immune response. An effective treatment strategy for periprosthetic joint infection (PJI) has long been debated. Bacterial biofilm communities provide constituent bacteria substantial protection against antimicrobial agents and the host immune response; thus, bacterial biofilm pose a major challenge in treating periprosthetic infections. Specifically, infectious bacteria readily form matrix-enclosed biofilm communities to evade phagocytic clearance by host neutrophils.

A phenotypic subset of biofilm cells can be tolerant of antibiotic concentrations that are many orders of magnitude greater than the antibiotic concentrations that would eliminate planktonic phenotypes. Biofilms are a focus of infection because biofilm persisters outlast antibiotic treatments to subsequently reseed infection. Furthermore, necrotic and devitalized tissue can represent sources of future infection. Physical removal of infectious biofilms and devitalized tissue by surgical debridement and hardware removal is thus used for the eradication of biofilm and associated infections.

### SUMMARY

In one embodiment, a method for removing non-viable tissue at a treatment situs can comprise administering a selective biofilm stain dye to the treatment situs at a dilution that selectively stains non-viable tissue compared to viable tissue at a selectivity ratio that provides a visually identifiable separation between the non-viable tissue and the viable tissue. In one aspect, the selective biofilm stain dye can be diluted in water to a concentration of from about 0.001 mg/mL to about 0.5 mg/mL. In another aspect, the method can comprise visually identifying the non-viable tissue. In one more aspect, the method can comprise removing the non-viable tissue based on the visual identification.

In another embodiment, a biofilm staining device can comprise an applicator. In one aspect, the applicator can include a dispense tip, a rupture lever, and a housing. In one aspect, the rupture lever can be adapted to rupture a rupturable capsule and release a selective biofilm stain dye. In another aspect, the housing can be sized to retain the rupturable capsule and adapted to allow released selective biofilm stain dye to flow to the dispense tip. In one aspect, the rupturable capsule can contain the selective biofilm stain dye at a dilution that selectively stains non-viable tissue compared to viable tissue at a selectivity ratio that provides a visually identifiable separation between the non-viable tissue and the viable tissue. In one aspect, the dilution can be from about 0.001 mg/mL to about 0.5 mg/mL.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure.

FIG. 1A is an illustration of a transmission light photometry setup for quantifying the methylene blue (MB) in stained biofilms. A transparent acrylic chamber filled with 0.9% saline is used to hold MB-stained biofilm coupons still mounted in a CDC reactor arms in accordance with an example.

FIG. 1B is an illustration of an acrylic chamber of FIG. 1A that reproducibly orients the coupons orthogonally between the light source and digital camera to quantify methylene blue (MB) binding by transmission light photometry in accordance with an example.

FIG. 1C is an illustration of a biofilm staining device including a rupturable capsule containing diluted methylene blue (MB) and an applicator including a porous dispense tip, a rupture lever, and a housing sized to retain the rupturable capsule in accordance with an example.

FIG. 1D is an illustration of a biofilm staining device including a rupturable capsule containing diluted methylene blue (MB) and an applicator including a porous dispense tip, a rupture lever, and a housing sized to retain the rupturable capsule in accordance with an example.

FIG. 2 is a photograph of S. aureus biofilms stained with methylene blue (MB) (Row 1) and imaged by scanning electron microscopy (SEM) (Row 2). Confirmation of the staphylococcal bacteria was confirmed by high resolution micrographs (Row 3) from the indicated representative regions (yellow arrowhead Row 2).

FIG. 3 shows P. aeruginosa biofilms stained with methylene blue (MB) (Row 1) and imaged by scanning electron microscopy (SEM) (Row 2). The monoculture of the rod-shaped bacteria was confirmed by high resolution micrographs (Row 3) from the indicated representative regions (yellow arrowhead Row 2).

FIG. 4 is a graph showing the binding of methylene blue (MB) to biofilms is both dose dependent and species dependent. The amount of MB bound per colony forming unit (CFU) is nearly an order of magnitude lower for S. aureus biofilms when compared with P. aeruginosa biofilms. The error bars represent the S.D. (n=6) and the bracket insets give the p-value as calculated by a Student's T-test.

FIGS. 5A and 5B illustrate that methylene blue (MB) does not stain orthopedic implant biomaterials. A) Biomaterials shown without visual evidence of blue staining. B) Histogram showing digital analysis of blue staining. There is no difference between controls and MB dilutions. The error bars represent the standard deviation within the zone of analysis. Data is normalized relative to unstained coupons. The y-axis represents perceived blueness expressed as a percent (%) methylene blue solution based on standardized dilutions seen at the bottom of each figure.

FIG. 6A and FIG. 6B also illustrate methylene blue (MB) does not stain most host tissue types and stains articular cartilage and meniscus in vitro. A) Tissues and controls shown. B) Histogram showing digital analysis of blue staining. Meniscus and cartilage show significant blue staining. The error bars represent the standard deviation within the zone of analysis. Data is normalized relative to unstained tissue. The y-axis represents perceived blueness expressed as a percent (%) methylene blue solution based on standardized dilutions seen at the bottom of each figure.

FIGS. 7A and 7B illustrate a serial dilution staining of fresh-frozen tissues with methylene blue (MB). (A) Fresh-frozen samples (˜1 cm³) of muscle and fat were stained with the indicated serial dilutions of MB for 5 min with 3 subsequent 1 min washes in excess 0.9% normal saline. (B) The perceived blueness of each photographed sample is represented by the average red channel intensity. The trace with circles is given for the muscle sample whereas the trace with squares is for fat tissue. There is an apparent inflection around 0.05 and 0.1 mg/ml where perceived blueness and red channel intensity rapidly change; these concentrations of methylene blue marked with an asterisk (*) were used in all other experimentation in this work.

FIG. 8 depicts a method for removing non-viable tissue at a treatment situs in accordance with an example.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a capsule” includes reference to one or more of such structures and reference to “applying” refers to one or more of such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, “selective” refers to modifying an action that provides a difference within a group (e.g., a group of cells) or between groups (e.g., a group of non-viable cells and a group of viable cells). For example, the action can be staining tissue and the groups can be non-viable tissue and viable tissue. For example, “selective staining” of non-viable tissue compared to viable tissue can provide a staining difference between the non-viable tissue and the viable tissue at a selectivity ratio. In one example, the selectivity ratio differs from a 1:1 ratio. In one example, the selectivity ratio can be a ratio that is greater than at least one of: 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 100:1, the like, and a combination thereof.

As used herein, the terms “treat,” “treatment,” or “treating” and the like refers to administration of a therapeutic agent or therapeutic action to a subject who is either asymptomatic or symptomatic. In other words, “treat,” “treatment,” or “treating” can refer to the act of reducing or eliminating a condition (i.e., symptoms manifested), or it can refer to prophylactic treatment (i.e., administering to a subject not manifesting symptoms in order to prevent their occurrence). Such prophylactic treatment can also be referred to as prevention of the condition, preventative action, preventative measures, and the like.

As used herein, a “treatment situs” refers to a location on or within a subject where treatment is desired. For example, when treating an infection, the treatment situs can be the area of the infection.

As used herein, a “subject” refers to a mammal that may benefit from the method or device disclosed herein. Examples of subjects include humans, and may also include other animals such as horses, pigs, cattle, dogs, cats, rabbits, and aquatic mammals. In one specific aspect, the subject is a human.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, diabetes, hypertension, high cholesterol, and the like.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

Occurrences of the phrase “in one embodiment,” or “in one aspect,” herein do not necessarily all refer to the same embodiment or aspect. Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

When treating hardware-associated infections, determining which tissue to remove and which tissue to leave is highly dependent on a surgeon's experience. This issue plays out in the literature relating to: (a) irrigation and debridement for acute PJI, and (b) single-stage versus two-stage exchange for chronic PJI. In both clinical scenarios, removing tenacious bacterial biofilms to reliably eradicate deep periprosthetic infections should be investigated.

However, there is little evidence to support a four-week time-based cutoff as a means of distinguishing between acute and chronic periprosthetic infections. As such, the different treatments for acute and chronic infections are difficult to support. For periprosthetic infections caused by biofilm forming organisms, such as Staphylococcal species or Pseudomonas, there is no literature to support periprosthetic infection without biofilm. For example, techniques such as irrigation and debridement used to treat acute PJI have often resulted in a high rate of clinical failures. Nonetheless, these techniques (irrigation and debridement) have persisted because two-stage exchange to achieve infection control has been perceived as undesirable. Furthermore, the infection rate with single-stage exchange revision is roughly twice that found with a two-stage exchange (12.3% vs 6.5%). Because of this disparity between single stage and two-stage exchange, the single-stage technique is infrequently used because of concern about residual biofilm on host tissue after the initial resection and debridement.

Currently, there are no validated techniques to visualize bacterial biofilms in orthopedic applications. Because residual biofilm on both implant and host tissue may be the common mode of failure for resolving deep periprosthetic infections, a technique to reliably identify biofilm in the operative settings can reduce the failure rate and consequent morbidity, mortality, and cost. Adequate debridement, however, is complicated by the inability to visualize biofilms, necrotic tissue, and devitalized tissue with the naked eye.

Furthermore, visualizing biofilms and necrotic tissue with the naked eye can provide an adequate visual indication between necrotic tissue and viable tissue when the visualization technique provides sufficient differentiation between the necrotic tissue that should be removed and the otherwise healthy tissue that should remain. Unclear or non-selective staining can be more harmful than relying on a surgeon's overall level of ability. Therefore, a useful staining technique should prove a sufficiently selective differentiation between healthy tissue and necrotic tissue in a surgical context. Removing too much healthy tissue or removing inadequate amounts of non-viable tissue can impact subject viability. Therefore, enhanced accuracy (e.g., staining non-viable tissue to the exclusion of other tissue) and enhanced precision (e.g., a reduction in the variability of staining) in staining can increase the effectiveness of patient surgical outcomes.

Consequently, a method for removing non-viable tissue at a treatment situs can include administering a selective biofilm stain dye to the treatment situs at a dilution that selectively stains non-viable tissue compared to viable tissue at a selectivity ratio that provides a visually identifiable separation between the non-viable tissue and the viable tissue. In one aspect, the selective biofilm stain dye can be diluted in water to a concentration of from about 0.001 mg/mL to about 0.5 mg/mL (i.e. 0.0001% to 0.05% by volume), and in some cases about 0.002 mg/mL to about 0.05 mg/mL. In still another alternative, the concentration can be up to about 0.1 mg/mL. In another aspect, the method can include visually identifying the non-viable tissue. In yet another aspect, the method can comprise removing the non-viable tissue based on the visual identification.

In another embodiment, a biofilm staining device can comprise an applicator including: a dispense tip, a rupture lever adapted to rupture a rupturable capsule and release a selective biofilm stain dye, and a housing sized to retain the rupturable capsule and adapted to allow released selective biofilm stain dye to flow to the dispense tip. In one aspect, the rupturable capsule can contain the selective biofilm stain dye at a dilution that selectively stains non-viable tissue compared to viable tissue at a selectivity ratio that provides a visually identifiable separation between the non-viable tissue and the viable tissue, wherein the dilution is from about 0.001 mg/mL to about 0.5 mg/mL (i.e. 0.0001% to 0.05% by volume), and in some cases about 0.002 mg/mL to about 0.05 mg/mL. In still another alternative, the dilution can be up to about 0.1 mg/mL.

A diluted selective biofilm stain dye can be used as a biofilm disclosing agent in vitro for common biofilm forming organisms to determine performance characteristics across implant materials and healthy tissue types. As an example, methylene blue (MB) is a phenothiazine compound that works both as a monoamine oxidase inhibitor and as an effective staining dye. Diluted methylene blue solution has been found to bind and stain bacterial biofilms and non-viable/necrotic tissue. Bacterial biofilms are typically invisible to the naked eye. Staining with diluted methylene blue allows for visualization of bacterial biofilms and, once visualized, they can be eradicated.

Various selective biofilm stain dyes can be used as biofilm disclosing agents. A selective biofilm stain dye can include but is not limited to methylene blue, isosulfan blue, plaque disclosing agent, deep green dye, and a combination thereof. Non-limiting examples of specific dyes can include fluoresciene, FD&C yellow 8 w/UV light, fast green, brilliant green, FD&C Green No 3, Phyloxine B, eosine, FD&C Red No. 40, erythrosin, FD&C Red No. 3, Gentian violet, Crystal violet, and Indocyanine green with laser illumination. In one specific example, the selective biofilm stain dye can be methylene blue.

In one embodiment, the selective biofilm stain dye (e.g., methylene blue), when diluted with water, can be present in a concentration of from about 0.001 mg/mL to about 0.5 mg/mL. In one example, the selective biofilm stain dye (e.g., methylene blue) can be present in a concentration of from about 0.01 mg/mL to about 0.1 mg/mL. In another example, the selective biofilm stain dye (e.g., methylene blue) can be present in a concentration of from about 0.005 mg/mL to about 0.025 mg/mL. In another example, the selective biofilm stain dye (e.g., methylene blue) can be present in a concentration of from about 0.002 mg/mL to about 0.01 mg/mL.

In another embodiment, diluting the selective biofilm stain dye (e.g., methylene blue) with water at a selected concentration can provide a non-viable tissue to viable tissue selectivity ratio that provides a visually identifiable separation between the non-viable tissue and the viable tissue.

In one embodiment, the selective biofilm stain dye can be diluted with water to provide a selectivity ratio for staining non-viable tissue compared to staining viable tissue of greater than one or more of: 1:10, 1:15, 1:20, 1:25, 1:30, 1:35, 1:40, 1:45, 1:50, the like, or combinations thereof. In another example, the selective biofilm stain dye can be diluted with water to provide a selectivity ratio for staining non-viable tissue compared to staining viable tissue of greater than one or more of 1:10, 1:12, 1:14, 1:16, 1:18, 1:20, 1.22, 1:25, the like, or combinations thereof.

In another embodiment, diluting the selective biofilm stain dye (e.g., methylene blue) with water at a selected concentration can provide a non-viable tissue to implanted tissue selectivity ratio that provides a visually identifiable separation between the non-viable tissue and the implanted tissue.

In one embodiment, the selective biofilm stain dye can be diluted with water to provide a selectivity ratio for staining non-viable tissue compared to staining implanted material of greater than one or more of: 1:10, 1:15, 1:20, 1:25, 1:30, 1:35, 1:40, 1:45, 1:50, the like, or combinations thereof. In another example, the selective biofilm stain dye can be diluted with water to provide a selectivity ratio for staining non-viable tissue compared to staining implanted material of greater than one or more of 1:10, 1:12, 1:14, 1:16, 1:18, 1:20, 1:22, 1:25, the like, or combinations thereof.

In yet another embodiment, diluting the selective biofilm stain dye (e.g., methylene blue) with water at a selected concentration can provide an extracellular location to intracellular location selectivity ratio that provides a visually identifiable separation between the extracellular location and the intracellular location. In some cases, the water dilution can take the form of a normal saline solution.

In one embodiment, the selective biofilm stain dye can be diluted with water to provide a selectivity ratio for staining extracellular locations compared to staining intracellular locations of greater than one or more of: 1:10, 1:15, 1:20, 1:25, 1:30, 1:35, 1:40, 1:45, 1:50, the like, or combinations thereof. In another example, the selective biofilm stain dye can be diluted with water to provide a selectivity ratio for staining extracellular locations compared to staining intracellular locations of greater than one or more of 1:10, 1:12, 1:14, 1:16, 1:18, 1:20, 1:22, 1:25, the like, or combinations thereof.

In one more embodiment, diluting the selective biofilm stain dye (e.g., methylene blue) with water at a selected concentration can provide a persister cell to viable tissue selectivity ratio that provides a visually identifiable separation between the persister cells and the viable tissue.

In one embodiment, the selective biofilm stain dye can be diluted with water to provide a selectivity ratio for staining persister cells compared to staining viable tissue of greater than one or more of 1:10, 1:15, 1:20, 1:25, 1:30, 1:35, 1:40, 1:45, 1:50, the like, or combinations thereof. In another example, the selective biofilm stain dye can be diluted with water to provide a selectivity ratio for staining persister cells compared to staining viable tissue of greater than one or more of 1:10, 1:12, 1:14, 1:16, 1:18, 1:20, 1:22, 1:25, the like, or combinations thereof.

The implanted material can comprise various materials including but not limited to titanium, cobalt, chromium, stainless steel, carbon, diamond like carbon, titanium niobium nitride, tantalum, polyethylene, ultra high molecular weight polyethylene, polyetheretherketone (PEEK), the like, or combinations thereof. In another example, the implanted material can comprise a material including but not limited to polyvinylchloride (PVC), polypropylene (PP), polymethylmethacrylate (PMMA), calcium sulfate, polystyrene (PS), polytetrafluoroethylene (PTFE), polyurethane (PU), polyamide (nylon), polyethyleneterephthalate (PET), polyethersulfone (PES), polyetherimide (PEI), polyester, polycarbonate, the like, and combinations thereof. In another example, the implanted material can comprise a material including but not limited to stainless steel, cobalt-chrome alloy, nickel-titanium alloy, gold, platinum, silver, iridium, tantalum, tungsten, the like, and combinations thereof.

In one example, the biofilms can be one or more of: gram-positive bacteria (e.g. Bacillus spp, Listeria monocytogenes, Staphylococcus spp, Lactobacillus plantarum, and Lactococcus lactis, Enterococcus spp), gram-negative bacteria (e.g. Escherichia coli, Acinetobacter baumannii, Klebsiella pneumoniae, or Pseudomonas aeruginosa), cyanobacteria, archaea, fungi, microalgae, anaerobes, aerobes, facultative aerobes, the like, or combinations thereof. Some representative biofilm organisms include: Pseudomonas aeruginosa, Streptococcus pneumoniae, Streptococcus pyogenes, Escherichia coli, methicillin-resistant Staphylococcus aureus (MRSA), Staphylococcus aureus ATCC 6538, Pseudomonas aeruginosa ATCC 27853, Cutibacterium acnes, Staphylococcus epidermidis, the like, or combinations thereof.

In one embodiment, the non-viable tissue can be visually identified by a practitioner after staining. The visual identification can be conducted by a practitioner without the assistance of other devices or in conjunction with various operations including creating a visual image of the subject, analyzing and/or processing the visual images of the subject, pre-surgical planning, surgical navigation, robotic surgery, the like, or combinations thereof. The staining can be integrated with different medical imaging technologies to create the visual image such as computed tomography (CT) scan, magnetic resonance imaging (MRI), x-rays, ultrasound, the like, or combinations thereof. The staining and the medical imaging technologies can create a 3D dataset to reproduce the geometry of viable tissue, non-viable tissue, implanted locations, extracellular locations, intracellular locations, persister cells, and the like. The 3D dataset can be modeled using different contrast levels for various types tissue, locations, or cells to create a virtual 3D model. The 3D model can be used by a practitioner to visually identify the non-viable tissue and to remove the non-viable tissue.

In one embodiment, the non-viable tissue can be removed using any suitable technique. In one example, the suitable technique can be surgical mechanical debridement using scalpels, curettes, scissors, the like, or combinations thereof. In another embodiment, the suitable technique can include but is not limited to amputation, resection, segmental resection, excision, extirpation, the like, or combinations thereof. In another embodiment, the suitable technique can include mechanical debridement including but not limited to hydrotherapy, directed wound irrigation, therapeutic irrigation with suction, wet-to-dry dressing, monofilament debridement pads, the like, or combinations thereof. In another embodiment, the suitable techniques can be biological debridement (e.g., maggot debridement), enzymatic debridement, autolytic debridement, the like, or combinations thereof.

A diluted selective biofilm stain dye (e.g., diluted methylene blue dye) can have broad applications in the treatment of biofilm associated infections requiring debridement. In one embodiment, the treatment situs can be at least one of: a skin infection, a soft tissue infection, periprosthetic joint infections, deep spine implant associated infections, orthopedic hardware associated infections, osteomyelitis, infected non-union, infected prosthetic heart valves, infected prosthetic stents, indwelling catheter associated infections, routine stoma maintenance for indwelling prosthetic devices, and a combination thereof.

In one embodiment, the selective biofilm stain dye can be substantially free of antiseptics, antibiotics, antivirals, antifungals, antiprotozoan agents, the like, or combinations thereof. Alternatively, the selective biofilm stain dye can include one or more of such materials. Non-limiting examples can include amebicides such as chloroquine, nitazoxanide, paromomycin, tinidazole, metronidazole, iodoquinole, or the like; aminoglycosides such as tobramycin, gentamicin, amikacin, kanamycin, neomycin, streptomycin, or the like; anthelmintics such as albendazole, ivermectin, praziquantel, pyrantel, mebendazole, miltefosine, niclosamide, piperazine, thiabendazole, or the like; antifungals such as itraconazole, posaconazole, ketoconazole, fluconazole, clotrimazole, isavuconazole, miconazole, voriconazole, echinocandins, terbinafine, griseofulvin, flucytosine, nystatin, amphotericin b, or the like; antimalarials such as chloroquine, quinine, hydroxychloroquine, mefloquine, primaquine, pyrimethamine, halofantrine, doxycycline, or the like; antituberculosis agents such as aminosalicylic acid, bedaquiline, isoniazid, ethambutol, pyrazinamide, ethionamide, rifampin, rifabutin, rifapentine, capreomycin, cycloserine, streptomycin, or the like; antivirals such as amantadine, rimantadine, ritonavir, cobicistat, peginterferon alfa-2a, peginterferon alfa 2b, maraviroc, raltegravir, dolutegravir, elvitegravir, sofosbuvir, enfuvirtide, fomivirsen, foscarnet, oseltamivir, zanamivir, peramivir, etravirine, efavirenz, nevirapine, delavirdine, rilpivirine, daclatasvir, adefovir, entecavir, telbivudine, didanosine, tenofovir, abacavir, lamivudine, zidovudine, stavudine, emtricitabine, zalcitabine, boceprevir, simeprevir, fosamprenavir, lopinavir, darunavir, telaprevir, ritonavir, tipranavir, atazanavir, nelfinavir, amprenavir, indinavir, saquinavir, ganciclovir, valacyclovir, famciclovir, acyclovir, valganciclovir, ribavirin, cidofovir, or the like; carbapenems such as doripenem, meropenem, cilastatin, ertapenem, or the like; cephalosporins such as avibactam, ceftolozane, ceftazidime, tazobactam, cefadroxil, cephalexin, cefazolin, ceftaroline, loracarbef, cefotetan, cefuroxime, cefprozil, cefaclor, cefoxitin, ceftibuten, cefotaxime, ceftriaxone, cefpodoxime, cefixime, cefdinir, defditoren, ceftazidime, ceftizoxime, cefepime, or the like; glycopeptide antibiotics such as vancomycin, dalbavancin, oritavancin, telavancin, or the like; glycocyclines such as tigecycline, or the like; leprostatics such as thalidomide, dapsone, clofazimine, or the like; lincomycin, or the like; clindamycin, or the like; ketolides such as telithromycin, or the like; macrolides such as azithromycin, fidaxomicin, erythromycin, clarithromycin, or the like; antibiotics such as aztreonam, daptomycin, chloramphenicol, colistimethate, fosfomycin, rifaximin, metronidazole, sulfamethoxazole, atovaquone, bacitracin, dalfopristin, erythromycin, furazolidone, pentamidine, polymyxin b, spectinomycin, trimetrexate, linezolid, tedizolid, penicillins (e.g. ampicillin, amoxicillin, carbenicillin, piperacillin, ticarcillin, nafcillin, dicloxacillin, cloxacillin, oxacillin, or the like), quinolones (e.g. lomefloxacin, norfloxacin, ofloxacin, gatifloxacin, moxifloxacin, ciprofloxacin, levofloxacin, gemifloxacin, cinoxacin, nalidixic acid, sparfloxacin, or the like), sulfonamides (e.g. sulfamethoxazole, sulfadiazine, sulfisoxazole, or the like), tetracyclines (e.g. tetracycline, demeclocycline, doxycycline, minocycline, or the like), or the like; urinary anti-infectives such as methenamine, fosfomycin, nitrofurantoin, trimethoprim, cinoxacin, nalidixic acid, oxytetracycline, or the like; hydrates thereof, acids thereof, bases thereof, salts thereof, or combinations of any of such anti-infective agents.

The diluted selective biofilm stain dye (e.g., diluted methylene blue dye) can comprise various additives including but not limited to: pH buffers (e.g. to form an isotonic solution), phosphate buffers, normal saline 0.9%, adjusted saline or electrolyte solution (e.g., to form osmotically balanced solutions), sterile water for irrigation, sterile saline for irrigation, irrigation solutions with additives including antimicrobial compounds, the like, or combinations thereof.

The diluted selective biofilm stain dye (e.g., diluted methylene blue dye) can be administered to a subject using any appropriate device. In one example, a biofilm staining device can comprise a rupturable capsule and an applicator. The rupturable capsule can contain the diluted selective biofilm stain dye (e.g., diluted methylene blue dye). The diluted selective biofilm stain dye (e.g., diluted methylene blue dye) can be present at a dilution that selectively stains non-viable tissue compared to viable tissue at a selectivity ratio that provides a visually identifiable separation between the non-viable tissue and the viable tissue. In one aspect, the dilution can be from about 0.002 mg/mL to about 0.05 mg/mL, or any other dilution disclosed herein.

The applicator can include a dispense tip, a rupture lever, and a housing. The rupture lever can be adapted to rupture the rupturable capsule and release the diluted selective biofilm stain dye (e.g., diluted methylene blue dye). The housing can be sized to retain the rupturable capsule and adapted to allow released diluted selective biofilm stain dye (e.g., diluted methylene blue dye) to flow to the dispense tip. In one aspect, the dispense tip can further comprise a porous dispense surface adapted to receive, retain, and release the diluted selective biofilm stain dye (e.g., diluted methylene blue dye) to a treatment situs.

In one example, a method of using the biofilm staining device can include activating the rupture lever to release the diluted selective biofilm stain dye (e.g., diluted methylene blue dye). The dispense tip can be contacted with target tissue including suspected bacterial biofilm such that the diluted selective biofilm stain dye (e.g., diluted methylene blue dye) selectively stains the bacterial biofilm differentially greater than surrounding healthy tissue. The operator can then visually identify the stained bacterial biofilm by viewing the differential staining. The stained bacterial biofilm can then be removed, e.g., by resection and/or debridement of stained bacterial biofilm.

Furthermore, the diluted selective biofilm stain dye (e.g., diluted methylene blue dye) can be targeted at extracellular locations, as opposed to within cells. The diluted selective biofilm stain dye (e.g., diluted methylene blue dye) can also be targeted to persister cells as opposed to viable tissue, or to non-viable tissue as opposed to implanted material. The diluted selective biofilm stain dye (e.g., diluted methylene blue dye) can also be targeted to bacterial biofilm including at least one of: Staphylococcus aureus, Pseudomonas aeruginosa, the like, and a combination thereof as opposed to healthy tissue. In another example, the diluted selective biofilm stain dye (e.g., diluted methylene blue dye) can also be targeted to biofilms including gram-positive bacteria (e.g., Bacillus spp, Listeria monocytogenes, Staphylococcus spp, Lactobacillus plantarum, and Lactococcus lactis), gram-negative bacteria (e.g. Escherichia coli, or Pseudomonas aeruginosa), cyanobacteria, archaea, fungi, microalgae, the like, or a combination thereof.

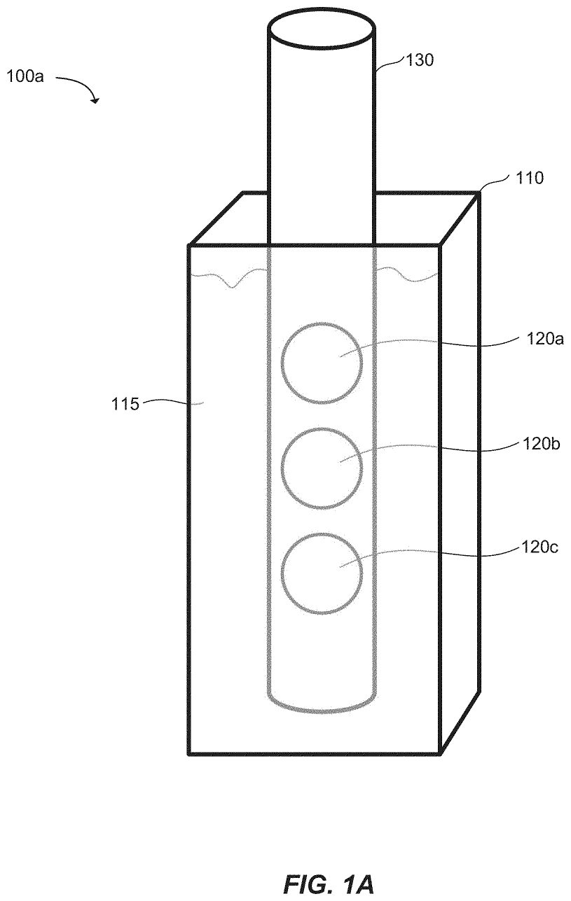

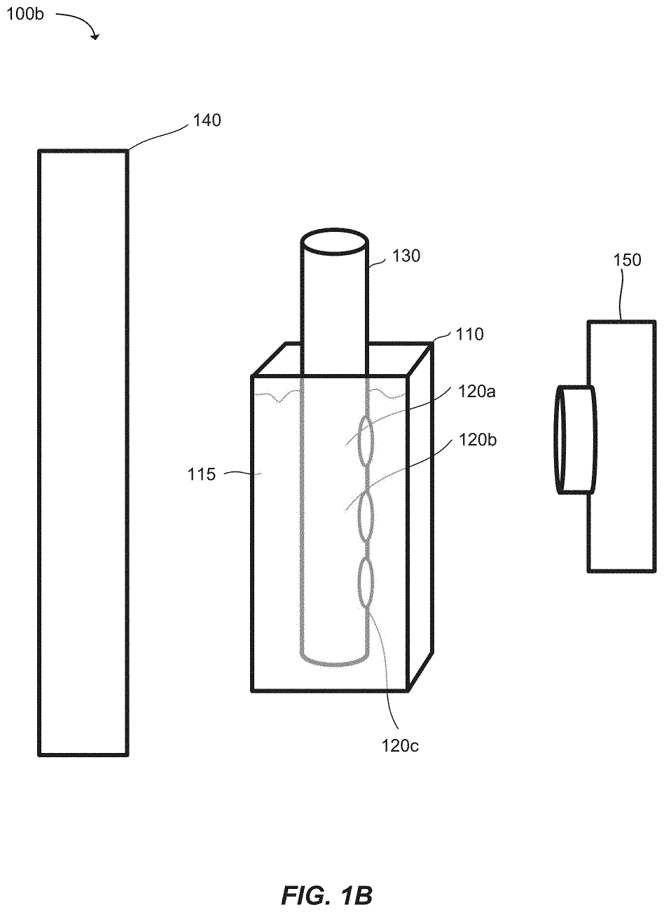

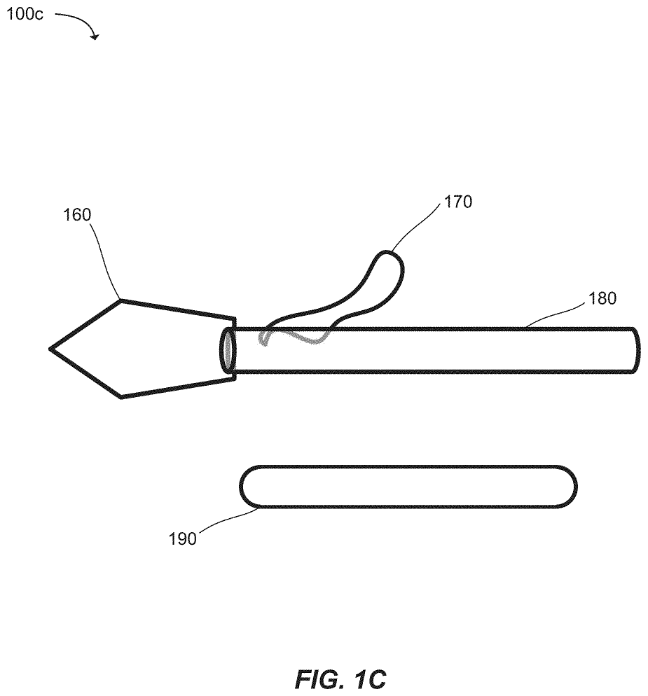

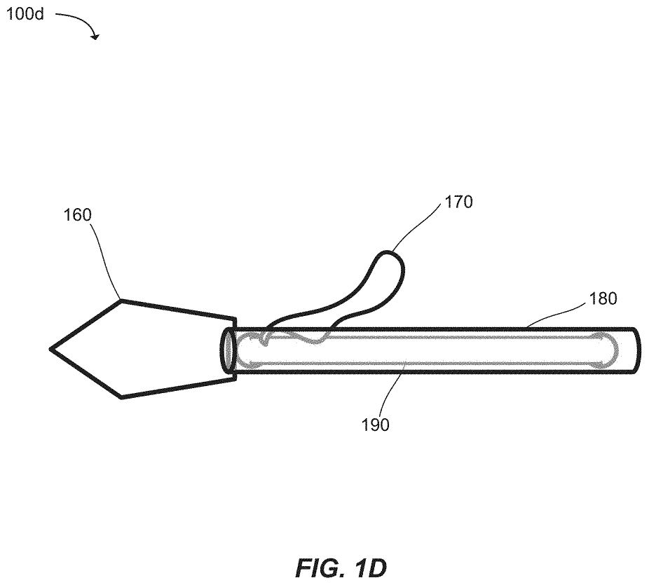

In one embodiment, as illustrated in FIG. 1A, a transmission light photometry setup 100a for quantifying the methylene blue (MB) in stained biofilms can comprise a transparent acrylic chamber 110 filled with 0.9% saline 115 is used to hold MB-stained biofilm coupons 120a, 120b, 120c still mounted in the CDC reactor arms 130. As illustrated in FIG. 1B, an acrylic chamber 110 can reproducibly orient the coupons 120a, 120b, 120c orthogonally between the light source 140 and digital camera 150 to quantify methylene blue (MB) binding by transmission light photometry in the setup 100b.

The biofilm staining device can allow precise delivery of dilute methylene blue solution specifically or diluted selective biofilm stain dye more generally. The device 100c, as illustrated in FIG. 1C, can include a sterile plastic tube 180 filled with glass cylinder(s) 190 containing sterile diluted methylene blue solution or diluted selective biofilm stain dye. The plastic tube 180 can be crimped with attached plastic lever 170, this action can break the glass cylinder(s) 190 and release the blue dye or diluted selective biofilm stain dye. The blue dye or diluted selective biofilm stain dye can then be applied to human tissues via a foam applicator tip 160. Glass shards can be retained via an internal filter mechanism (not shown). As illustrated in FIG. 1D, the device 100d can include a plastic tube 180 that encloses the glass cylinder 190.

Furthermore, the rupturable capsule 190 can include a dye solution having a volume of about 8 cm³ to about 30 cm³, and in some cases 15 cm³ to 22 cm³, although other volumes may be suitable for specific procedures. The dispense tip 160 can also include a porous dispense surface such as a sponge.

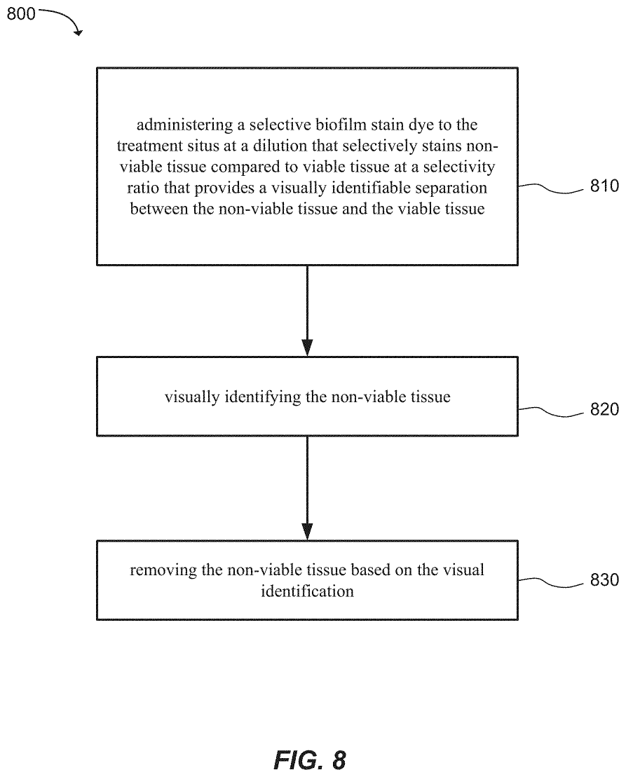

FIG. 8 illustrates a flow diagram of a method according to the present technology. For simplicity of explanation, the method is depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter.

In one example, a method 800 for removing non-viable tissue at a treatment situs is provided. The method can include administering a selective biofilm stain dye to the treatment situs at a dilution that selectively stains non-viable tissue compared to viable tissue at a selectivity ratio that provides a visually identifiable separation between the non-viable tissue and the viable tissue, wherein the selective biofilm stain dye is diluted in water to a concentration of from about 0.002 mg/mL to about 0.05 mg/mL, as shown in block 810. The method can further include visually identifying the non-viable tissue, as shown in block 820. The method can further include removing the non-viable tissue based on the visual identification, as shown in block 830.

### Experimental Example

A biofilm disclosing agent can be used in vitro for common biofilm forming organisms and can effectively differentially stain bacterial biofilm over various implant materials and healthy tissue types.

Sample Preparation.

Staphylococcus aureus (ATCC 6538) and Pseudomonas aeruginosa (ATCC 27853) biofilms were grown for the respective experiments (2 days) in a CBR 90 CDC biofilm reactor (BioSurface Technologies Corporation, Bozeman, Mont.) on either titanium, cobalt chromium, polyether ether ketone (PEEK), and polyethylene (PE) coupons using previously established methods. Briefly, a 0.5 McFarland standard (˜7.5×10⁷ colony forming units (CFU)) was made from a 24 hour tryptic soy agar plate culture; 1 mL was then used to inoculate 500 mL of 100% Brain Heart Infusion (BHI) broth in the biofilm reactor. The reactor and its contents were incubated at 34° C. on a hot plate with baffle rotation for 24 hours. After 24 hours, the reactor was flushed continuously with 10% BHI at 6.94 mL/min for the subsequent 24 hours. Prior to subsequent work, the biofilm coupons were removed aseptically and rinsed in PBS. Quantification of biofilm bioburden was performed on representative coupons from each reactor by dispersing the respective biofilm into 2 mL of fresh 1× phosphate buffered saline (PBS) following an established protocol: sequentially vortexing for 1 minute, sonicating for 10 minutes and vortexing for seconds. The CFUs were calculated by 10-fold serial dilution plating and colony counting.

For comparison, other common materials in the orthopedic wound sites were analyzed alone, without attached biofilms. Clean coupons of orthopedic relevant biomaterials were acquired from BioSurface Technologies Corporation: titanium, cobalt chromium, polyethylene and PEEK. Representative tissue samples were sourced from healthy female adult Rambouillet sheep hindlimbs: articular cartilage, meniscus, bone, tendon, muscle, nerve, and fat (under proper IACUC regulations). Tissues were harvested by a fellowship trained orthopedic surgeon.

Sample Staining.

Samples were stained in triplicate with 0.005% (1:100 of a 0.5% stock solution) and 0.01% (1:50 of a 0.5% stock solution) MB solutions for 5 minutes. Each sample was then washed 3 times for 1 minute each in excess normal saline prior to subsequent analysis. Representative samples were selected for imaging.

Photography:

The red channel intensity of stained orthopedic materials and tissues was analyzed to determine the perceived blueness of both control and MB stained samples; an increase in blueness due to MB binding is observed as attenuation of the red channel intensity; MB has two absorbance peaks in the visible range at 609 and 668 nm, both within the red spectral region (625-740 nm). ImageJ software was used to isolate the sample regions in the photographs and calculate the average red channel intensity within.

Scanning Electron Microscopy:

To validate that the blue stain corresponded with the presence of biofilm, after digital images were collected, biofilm coupons were fixed in modified Kamovsky's fixative (2 hours), dehydrated in ascending concentrations of 70%, 95% and 100% EtOH (1 hour each), gold sputter coated, and imaged in a JEOL JSM-6100 tungsten filament scanning electron microscope (SEM). To match the magnification of the digitally coupon photograph four SEM images were mosaiced using phase correlation image registration. Representative high magnification images (4300×) were also collected for each biofilm coupon.

Transmission Light Photometry:

Transmission photometry was used to determine the amount of MB bound to biofilms grown and stained using the above protocols. Biofilms were grown on transparent polycarbonate coupons to enable the transmission light analysis. A transparent acrylic chamber was designed to hold a CDC biofilm reactor arm and mounted coupons, orthogonally between the light source and a digital camera serving as a light sensor, as illustrated in FIGS. 1A to 1C. The exposure was fixed for all images analyzed; ambient light sources were eliminated by darkening the room. Setup conditions including light source, sample orientation, and camera position were held constant for all samples analyzed. Samples were imaged in 0.9% isotonic saline using the red channel (˜570-760 nm) and processed using an image J template to reproducibly isolate the three coupon regions of each reactor arm. Light transmittance was calculated by the average signal intensity measured through the coupon divided by the average signal intensity in the same region with the sample removed. Sample absorbance was then calculated using Beer's law by taking the Log₁₀ transform of the inverse of transmittance. Five coupon types were analyzed (n=6 for each group): clean without biofilm or MB stain, without biofilm but stained with 0.01% MB, unstained biofilms, biofilms stained in 0.01% MB, and biofilms stained in 0.005% MB. The absorbance attributed to the MB component for the MB-stained biofilms was found by subtracting the intrinsic absorbance for the unstained biofilm coupons. A calibration curve was created by filling the acrylic chamber of a known pathlength with a series of MB dilutions of known concentration. The total amount of MB in stained samples was thereby found by a linear regression from this calibration curve; and the number of MB molecules per CFU was computed from biofilm samples quantified as described above by serial dilution plating (n=6). The standard deviations were appropriately propagated for each computational operation. Descriptive statistics were used including means for continuous outcomes and proportions for categorical results. Statistical analysis was performed using SPSS Statistics software (IBM Corporation, New York, USA).

Results:

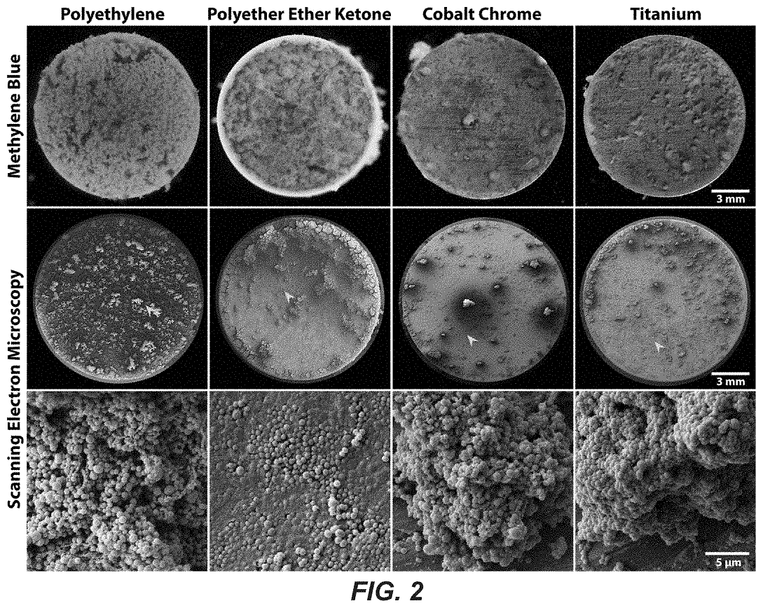

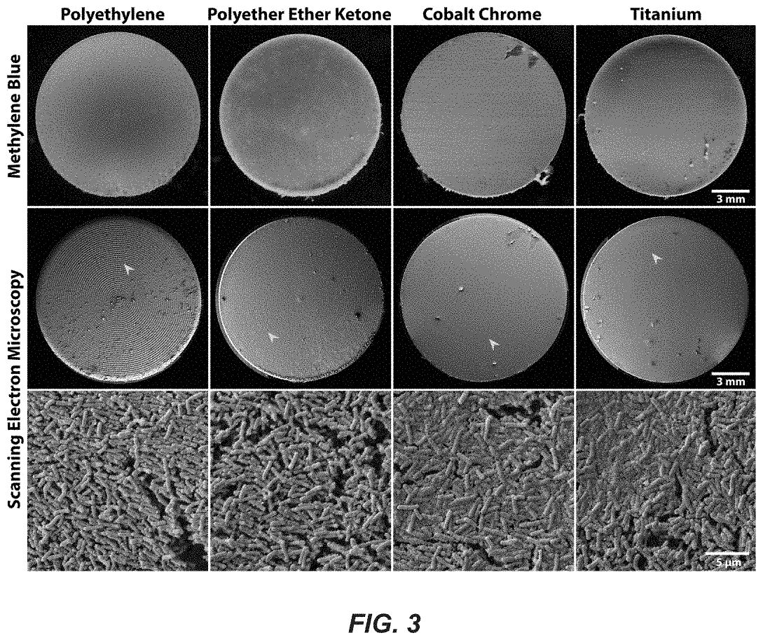

The S. aureus and P. aeruginosa biofilms grown on titanium, cobalt chromium, polyethylene, and PEEK coupons visually turned blue when treated with dilute MB, as illustrated in FIG. 2 & FIG. 3. MB staining was most visible where robust biofilms were present as confirmed by SEM, as illustrated in FIG. 2 and FIG. 3.

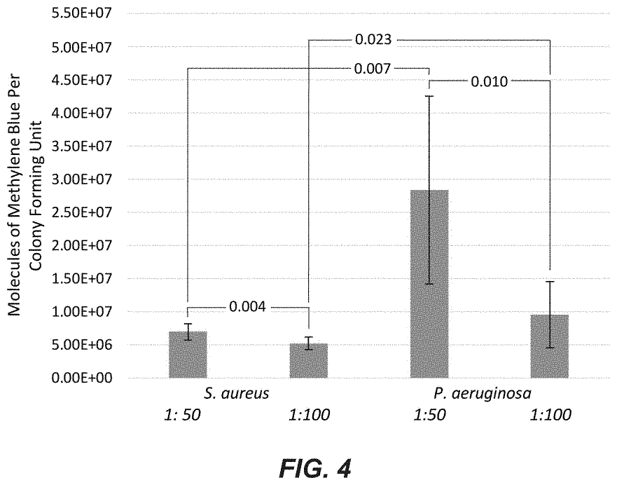

Concentration dependent staining was observed for both S. aureus and P. aeruginosa biofilms. Bacterial species-specific MB staining was also observed. At the highest MB concentration of 0.01% P. aeruginosa displayed nearly an order of magnitude increase in the number of MB molecules per CFU compared with S. aureus biofilms (2.84×10⁷ vs 6.95×10⁶ molecules/CFU, p<0.05), as illustrated in FIG. 4.

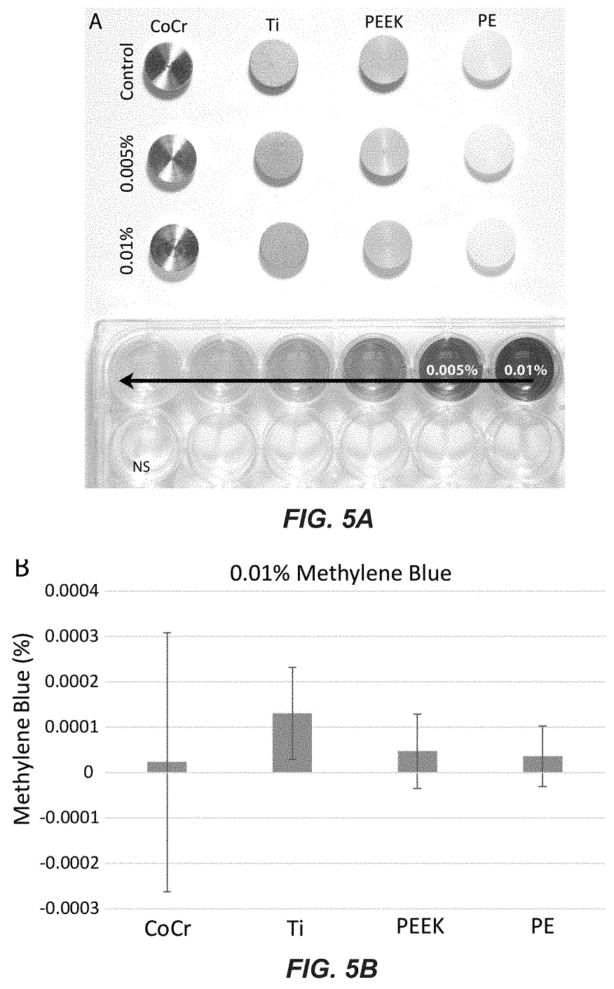

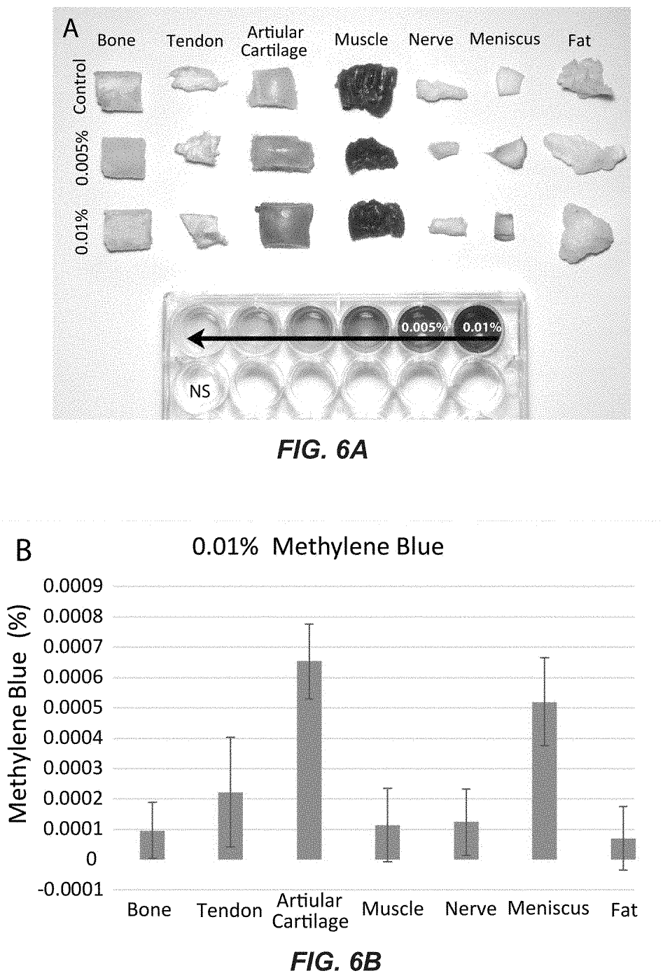

MB did not stain any implant materials, as shown in FIGS. 5A to 5B. This was evident visually and confirmed by digital analysis, as shown in FIG. 5B. Of healthy sheep tissues, articular cartilage and meniscus demonstrated significant staining; bone, tendon, muscle, nerve and fat did not display appreciable staining, as shown in FIGS. 6A to 6B. MB was visible where biofilms were present as confirmed by SEM. MB did not stain uninoculated controls. Bacterial biofilms demonstrated both dose-dependent and species-specific staining.

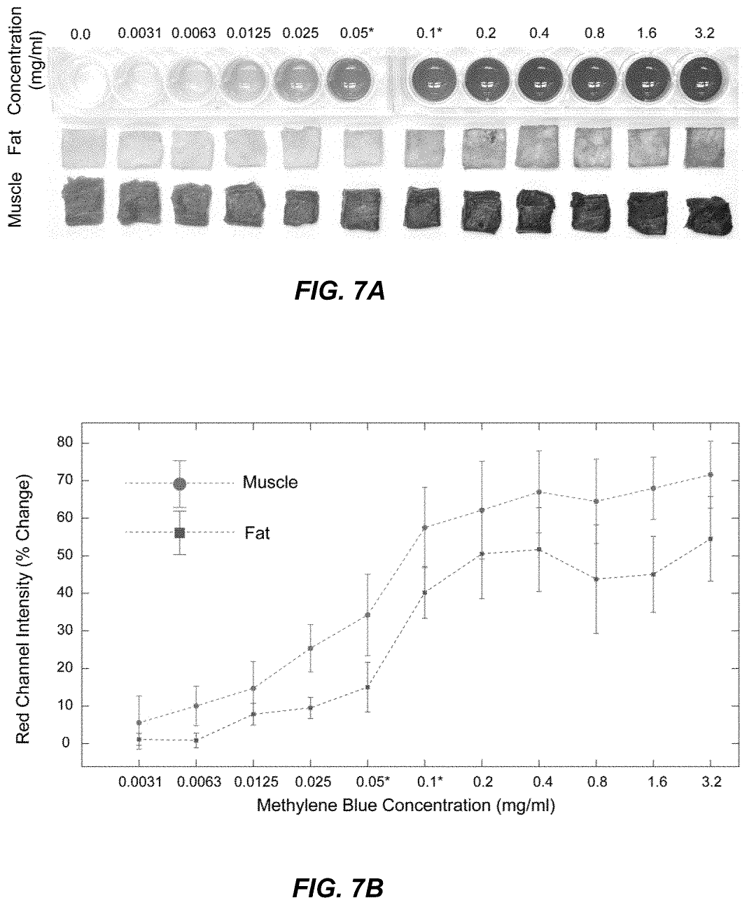

FIGS. 7A-7B illustrate a serial dilution staining of fresh-frozen tissues with methylene blue (MB). In FIG. 7A, fresh-frozen samples (˜1 cm³) of muscle and fat were stained with the indicated serial dilutions of MB for 5 min with 3 subsequent 1 min washes in excess 0.9% normal saline. In FIG. 7B the perceived blueness of each photographed sample is represented by the average red channel intensity. The trace with circles is given for the muscle sample whereas the trace with squares is for fat tissue. There is an apparent inflection around 0.05 and 0.1 mg/ml where perceived blueness and red channel intensity rapidly change; these concentrations of methylene blue marked with an asterisk (*) were used in all other experimentation in this work.

Discussion:

MB was assessed as a biofilm disclosing agent in vitro for common biofilm forming isolates and to determine staining characteristics across a range of implant materials and normal tissue types. MB is an effective disclosing agent for S. aureus and P. aeruginosa biofilms in vitro. MB did not stain titanium, cobalt chromium, polyethylene, or PEEK biomaterials without biofilm. Additionally, MB did not uniformly stain healthy tissues in vitro. Given these favorable performance characteristics in the lab, this technique can allow surgeons to see biofilm on implants and host tissues in the operating room.

One practical utility of this technique is to identify and eradicate biofilm. These methods also enable more effective irrigation and debridement, as well as single stage exchange for the management of deep periprosthetic infections. Potential applications extend beyond orthopedics and include cardiovascular, urologic, and neurosurgical domains where biofilm-based prosthetic infection remains challenging. For applications within the field of orthopedics, the potential of component retention, particularly for arthroplasty and spine minimizes the extreme patient morbidity and cost associated with two-stage exchanges.

MB can stain bacterial biofilms in vitro, it also can stain necrotic tissue. A dye exclusion test can be used to test MB staining to identify dead tissue. For example, live cells can process MB and dead cells are unable to efflux the blue dye.

One example used ovine tissue, as it was not possible or ethical to harvest fresh healthy human tissues. Ovine tissues have a high degree of homology with human tissues and are a large animal model for orthopedic applications. While MB does not stain all healthy ovine tissue subtypes, it does moderately stain articular cartilage and meniscus tissues. Both of these tissue types have high quantities of sulfated polysaccharides. Regardless of how effective this technique may be in clinical practice; it can be reserved for cases where native joint resection (total joint arthroplasty) or fusion (ankle or spinal arthrodesis) are the ultimate procedural goals. It is likely not suitable for treatment of infection in native joints where cartilage will be retained due to lack of differential staining in such tissue.

Orthopedic surgeons have used the term “persister cells” to describe microorganisms that persevere despite debridement and antimicrobial therapies. These organisms may use additional therapeutic measures including aggressive or novel chemotherapeutics and/or alternative approaches. The historical context of “persister cells”, is highly relevant, as the term describes cells in a biofilm(s) that are particularly tolerant to antibiotic regimens and thus compromise therapeutic approaches. While distinct, both types of “persister cells” are clinically detrimental and may use specific therapeutic approaches depending on the scenario.

Regardless, MB functions as an effective disclosing agent for S. aureus and P. aeruginosa biofilms in vitro. MB does not stain implants, nor does it uniformly stain healthy host tissues in vitro. Given in vitro success, MB may allow surgeons to see biofilms on implants and host tissue in vivo and in so doing, may allow for eradication of biofilms once visualized.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Reference throughout this specification to “an example” means that a particular feature, structure, or characteristic described in connection with the example is included in at least one embodiment of the present invention. Thus, appearances of the phrases “in an example” in various places throughout this specification are not necessarily all referring to the same embodiment.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A method for removing non-viable tissue at a treatment situs, comprising:
administering a selective biofilm stain dye to the treatment situs at a dilution that selectively stains non-viable tissue compared to viable tissue at a selectivity ratio that provides a visually identifiable separation between the non-viable tissue and the viable tissue, wherein the selective biofilm stain dye is diluted in water to a concentration of from about 0.001 mg/mL to about 0.5 mg/mL;
visually identifying the non-viable tissue; and
removing the non-viable tissue based on the visual identification.

2. The method of claim 1, wherein the selective biofilm stain dye is methylene blue, isosulfan blue, plaque disclosing agent, deep green dye, and a combination thereof.

3. The method of claim 1, wherein the selective biofilm stain dye is methylene blue.

4. The method of claim 1, wherein the selective biofilm stain dye is substantially free of antiseptics, antibiotics, antivirals, and antifungals.

5. The method of claim 1, wherein the selective biofilm stain dye includes at least one of: pH buffers, phosphate buffers, normal saline 0.9%, adjusted saline, electrolyte solution, sterile water for irrigation, sterile saline for irrigation, and a combination thereof.

6. The method of claim 1, wherein the concentration is about 0.002 mg/mL to about 0.05 mg/mL

7. The method of claim 1, wherein the selective biofilm stain dye selectively stains non-viable tissue compared to viable tissue at a selectivity ratio of greater than at least one of 10:1, 20:1, 30:1, 40:1, and 50:1, and a combination thereof.

8. The method of claim 1, wherein the selective biofilm stain dye selectively stains non-viable tissue compared to implanted material at a ratio of greater than at least one of: 10:1, 20:1, 30.1, 40:1, and 50:1, and a combination thereof.

9. The method of claim 8, wherein the implanted material comprises at least one of: titanium, cobalt, chromium, stainless steel, carbon, diamond like carbon, titanium niobium nitride, tantalum, polyethylene, polyetheretherketone (PEEK), polyvinylchloride (PVC), polypropylene (PP), polymethylmethacrylate (PMMA), calcium sulfate, polystyrene (PS), polytetrafluoroethylene (PTFE), polyurethane (PU), polyamide (nylon), polyethylenterephthalate (PET), polyethersulfone (PES), polyetherimide (PEI), polyester, polycarbonate, stainless steel, cobalt-chrome alloy, nickel-titanium alloy, gold, platinum, silver, iridium, tantalum, tungsten, and a combination thereof.

10. The method of claim 1, wherein the treatment situs is at least one of a skin infection, a soft tissue infection, periprosthetic joint infections, deep spine implant associated infections, orthopedic hardware associated infections, osteomyelitis, infected non-union, infected prosthetic heart valves, infected prosthetic stents, indwelling catheter associated infections, routine stoma maintenance for indwelling prosthetic devices, and a combination thereof.

11. The method of claim 1, further comprising:
removing the non-viable tissue using at least one of resection, debridement, and a combination thereof.

12. The method of claim 1, further comprising:
targeting the selective biofilm stain dye to extracellular locations compared to intracellular locations at a ratio of greater than at least one of: 10:1, 20:1, 30:1, 40:1, and 50:1, and a combination thereof.

13. The method of claim 1, wherein the selective biofilm stain dye selectively stains a bacterial biofilm including at least one of: Pseudomonas aeruginosa, Streptococcus pneumoniae, Streptococcus pyogenes, Escherichia coli, methicillin-resistant Staphylococcus aureus (MRSA), Staphylococcus aureus ATCC 6538, Pseudomonas aeruginosa ATCC 27853, Cutibacterium acnes, Staphylococcus epidermidis, and a combination thereof.

14. The method of claim 1, wherein the selective biofilm stain dye selectively stains persister cells compared to viable tissue at a ratio of greater than at least one of: 10:1, 20:1, 30:1, 40:1, and 50:1, and a combination thereof.

15. A biofilm staining device, comprising:
an applicator including:
a dispense tip,
a rupture lever adapted to rupture a rupturable capsule and release a selective biofilm stain dye, and
a housing sized to retain the rupturable capsule and adapted to allow released selective biofilm stain dye to flow to the dispense tip; and
the rupturable capsule containing the selective biofilm stain dye at a dilution that selectively stains non-viable tissue compared to viable tissue at a selectivity ratio that provides a visually identifiable separation between the non-viable tissue and the viable tissue, wherein the dilution is from about 0.002 mg/mL to about 0.05 mg/mL.

16. The biofilm staining device of claim 15, wherein the dispense tip further comprises a porous dispense surface adapted to receive, retain, and release the selective biofilm stain dye to a treatment situs.

17. The biofilm staining device of claim 15, wherein the selective biofilm stain dye is one of methylene blue, isosulfan blue, plaque disclosing agent, deep green dye, and a combination thereof.

18. The biofilm staining device of claim 15, wherein the selective biofilm stain dye is substantially free of antiseptics, antibiotics, antivirals, and antifungals.

19. The biofilm staining device of claim 15, wherein the selective biofilm stain dye selectively stains non-viable tissue compared to viable tissue at a selectivity ratio of greater than at least one of 10:1, 20:1, 30:1, 40:1, and 50:1, and a combination thereof.

20. The biofilm staining device of claim 15, wherein the selective biofilm stain dye selectively stains non-viable tissue compared to implanted material at a ratio of greater than at least one of: 10:1, 20:1, 30:1, 40:1, and 50:1, and a combination thereof.

21. The biofilm staining device of claim 15, wherein the selective biofilm stain dye selectively stains persister cells compared to viable tissue at a ratio of greater than at least one of: 10:1, 20:1, 30:1, 40:1, and 50:1, and a combination thereof.
